(19) United States
(12) Patent Application Publication
(10) Pub. No.: US 2013/0172650 A1
(43) Pub. Date: Jul. 4, 2013

(54) UPGRADING LIGHT OLEFINS

(71) Applicants: Shetian Liu, Bartlesville, OK (US); Jianhua Yao, Bartlesville, OK (US); Xiaochun Xu, Bartlesville, OK (US); Patrick W. Blosser, Owasso, OK (US); Joe D. Allison, Bartlesville, OK (US); Edward L. Sughrue, II, Bartlesville, OK (US)

(72) Inventors: Shetian Liu, Bartlesville, OK (US); Jianhua Yao, Bartlesville, OK (US); Xiaochun Xu, Bartlesville, OK (US); Patrick W. Blosser, Owasso, OK (US); Joe D. Allison, Bartlesville, OK (US); Edward L. Sughrue, II, Bartlesville, OK (US)

(73) Assignee: PHILLIPS 66 COMPANY, Houston, TX (US)

(21) Appl. No.: 13/717,869

(22) Filed: Dec. 18, 2012

Related U.S. Application Data
(60) Provisional application No. 61/583,073, filed on Jan. 4, 2012.

(51) Int. Cl. C07C 2/24

(52) U.S. Cl. CPC C07C 2/24
USPC 585/504; 585/520; 585/531; 585/530; 422/619

(57) ABSTRACT

Methods and systems relate to upgrading light olefins, such as ethylene, propylene and butylenes, diluted in a gas mixture, such as refinery fuel gas. The upgrading yields products in a gasoline, distillate, lube oil or wax range without requiring purification or compression of the gas mixture prior to feeding the gas mixture to a reactor. In operation, the mixture contacts a solid oligomerization catalyst, such as silica supported chromium, within the reactor. This contact occurs at a first temperature suitable to produce oligomers that are formed of the olefins and adsorb on the catalyst in liquid or solid phases. Next, heating the catalyst to a second temperature higher the first temperature desorbs the oligomers that are recovered and separated into the products.

### CROSS-REFERENCE TO RELATED APPLICATIONS

This application is a non-provisional application which claims the benefit of and priority to U.S. Provisional Application Ser. No. 61/583,073 filed Jan. 4, 2012, entitled “Upgrading Light Olefins,” which is hereby incorporated by reference in its entirety.

### STATEMENT REGARDING FEDERALLY SPONSORED RESEARCH OR DEVELOPMENT

None.

### FIELD OF THE INVENTION

Embodiments of the invention relate to olefin conversion at a first temperature followed by product recovery at a second temperature higher than the first temperature.

### BACKGROUND OF THE INVENTION

Fuel gas at refineries contains methane, hydrogen and light olefins, such as ethylene and propylene. The light olefins often provide more value if utilized for other purposes than burning as a constituent of the fuel gas. Exemplary prior applications include use of the propylene for feedstock in alkylation units to produce high-octane gasoline blend and use of the ethylene for making polymers.

However, these applications require separation of the ethylene and propylene that often each make up less than 20% of the fuel gas. Expensive cryogenic separations thus limit the value realized for the light olefins within the fuel gas. Further, some refineries due to distance from plastic manufacturing facilities lack a market for the ethylene.

Various catalysts exist for oligomerization of the light olefins into upgraded products. Previous techniques employed with these catalysts for the oligomerization rely on pure olefin feeds, solvent diluents and/or undesired operating conditions. The techniques fail to provide a viable solution for generating desired products using the fuel gas as feed and at limited pressure and temperature.

Therefore, a need exists for a method of upgrading light olefins into more valuable products.

### BRIEF SUMMARY OF THE DISCLOSURE

For some embodiments, a method of upgrading light olefins includes passing a feed stream containing the olefins with each between two and five carbons into contact at a first temperature with an oligomerization catalyst to form oligomers of the olefins. The method further includes recovering the oligomers by heating the catalyst to a second temperature higher than the first temperature. The heating releases from the catalyst the oligomers adsorbed on the catalyst when the catalyst and the olefins are contacted at the first temperature.

According to some embodiments, a method of upgrading light olefins includes contacting an oligomerization catalyst formed of at least one of chromium and nickel on a solid support with a fuel gas containing greater than 15% by volume methane and less than 75% by volume of the olefins that are at least one of ethylene, propylene and butylenes. The contacting at a pressure below 1400 kilopascals and at a first temperature between 20° C. and 300° C. produces oligomers that are formed of the olefins and adsorbed on the catalyst. Heating the catalyst to a second temperature between 200° C. and 500° C. and at least 50° C. higher than the first temperature desorbs the oligomers that are then separated into gasoline and distillate range products.

In some embodiments, a system for upgrading light olefins includes a reactor containing an oligomerization catalyst and coupled to a feed of olefins selected from at least one of ethylene, propylene and butylenes. The reactor configuration cycles between an oligomerization and adsorption operation in which the olefins and the catalyst are in fluid communication for contact at a first temperature and a recovery operation. For the recovery operation, heating to a second temperature higher than the first temperature desorbs from the catalyst oligomers of the olefins.

### BRIEF DESCRIPTION OF THE DRAWINGS

A more complete understanding of the present invention and benefits thereof may be acquired by referring to the following description taken in conjunction with the accompanying drawings.

FIG. 1 is a system schematic depicting operation of a reactor in an olefin oligomerization and adsorption step, according to one embodiment of the invention.

FIG. 2 is a system schematic depicting operation of the reactor in a subsequent product recovery step carried out at a higher temperature than the oligomerization and adsorption step, according to one embodiment of the invention.

FIG. 3 is a system schematic depicting operation of the reactor in a regeneration step that enables further recovering of upgraded products as shown in FIGS. 1 and 2, according to one embodiment of the invention.

### DETAILED DESCRIPTION

Turning now to the detailed description of preferred arrangements of the present invention, it should be understood that the inventive features and concepts may be manifested in other arrangements and that the scope of the invention is not limited to the embodiments described or illustrated.

Methods and systems relate to upgrading light olefins, such as ethylene, propylene and butylenes, diluted in a gas mixture, such as refinery fuel gas. The upgrading yields products in a gasoline, distillate, lube oil or wax range without requiring purification or compression of the gas mixture prior to feeding the gas mixture to a reactor. In operation, the mixture contacts a solid oligomerization catalyst, such as silica supported chromium, within the reactor. This contact occurs at a first temperature suitable to produce oligomers that are formed of the olefins and adsorb on the catalyst in liquid or solid phases. Next, heating the catalyst to a second temperature higher the first temperature desorbs the oligomers that are recovered and separated into the products.

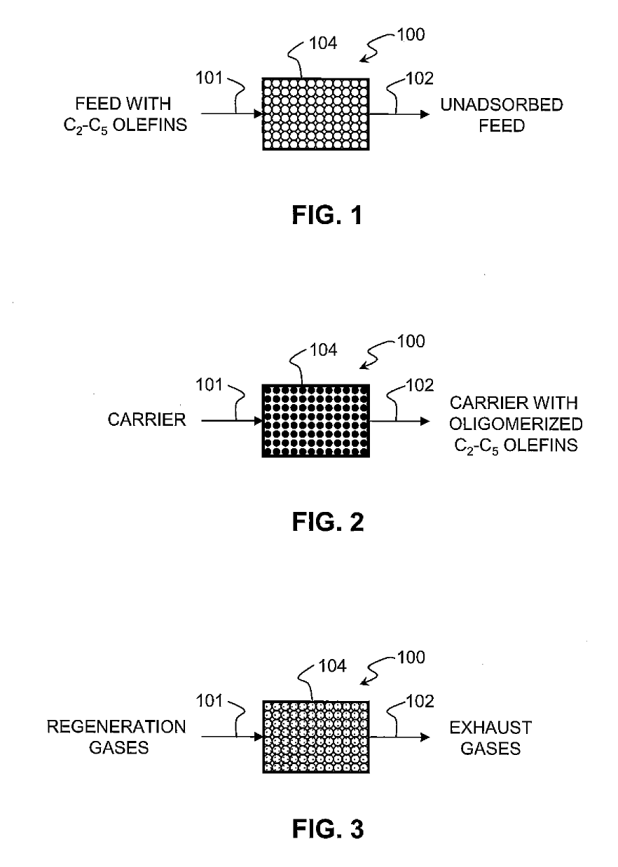

FIG. 1 illustrates a system schematic depicting operation of a reactor 100 in an olefin oligomerization and adsorption step. The reactor 100, such as a fixed bed reactor, includes an inlet 101 and an outlet 102. An oligomerization catalyst 104 fills an interior of the reactor 100 and is in fluid communication with the inlet 101 and the outlet 102 such that fluids entering the interior of the reactor 100 through the inlet 101 pass in contact with the catalyst 104 before exiting through the outlet 102.

Exemplary solid compositions suitable for the catalyst 104 used to oligomerize light olefins into oligomers for gasoline, distillate, lube oil and wax range products with between 5 and 30 carbons or 5 and 50 carbons include activated metals disposed on porous support materials. The catalysts 104 may include chromium, iron, cobalt, nickel and combinations thereof. Examples of the support materials for the catalyst 104 include silica and silica-alumina oxides.

In the oligomerization and adsorption step, a feed stream introduced through the inlet 101 contacts the catalyst 104, which is in an active state as depicted by unfilled circles in FIG. 1. The feed stream contains olefins selected from at least one of ethylene, propylene, butylenes and pentene and is in a gas phase when contacted with the catalyst 104. For some embodiments, fluid catalytic cracker (FCC) dry gas, coker light gas and/or refinery fuel gas forms the feed gas with any of these C2-C5 olefins.

The feed stream thus may include a mixture of gases with dilute ethylene. For example, the fuel gas suitable for the feed stream may contain greater than 15 percent by volume (vol. %) methane and less than 75 vol. % olefins. Exemplary concentrations within such feed streams range from 30 vol. % to 60 vol. % methane, 4 vol. % to 20 vol. % ethylene, 5 vol. % to 15 vol. % hydrogen, 0 vol. % to 15 vol. % propylene with remaining volume being other olefins with three or five carbons along with other gases.

The catalyst 104 and the feed stream contact at a first temperature during the oligomerization and adsorption step. For some embodiments, the first temperature ranges from 20° C. to 500° C. or from 20° C. to 300° C. Pressure in the reactor 100 for the oligomerization and adsorption step ranges in some embodiments from 100 kilopascals (kPa) to 7000 kPa or 100 kPa to 1400 kPa.

The oligomers of the olefins form as the feed stream contacts the catalyst 104. The oligomers deposit on surfaces of the catalyst 104 and are trapped in pores of the catalyst 104. Conditions in the reactor 100 throughout the oligomerization and adsorption step result in desirable products remaining in liquid and solid phases adsorbed on the catalyst 104. Given that selective oligomerization of the olefins occurs, non-olefin constituents of the feed stream do not react or get adsorbed and therefore pass through the reactor 100 and the outlet 102.

FIG. 2 shows a system schematic depicting operation of the reactor 100 in a subsequent product recovery step and the catalyst 104 in a loaded state as depicted by solid black filled circles. The oligomers adsorbed on the catalyst 104 tend to reduce catalyst activity as the oligomers increase during the oligomerization and adsorption step. The recovery step therefore releases the oligomers from the catalyst 104 by heating the catalyst 104 to a second temperature higher than the first temperature used in the oligomerization and adsorption step.

For some embodiments, the second temperature used in the recovery step exceeds the first temperature employed for the oligomerization and adsorption step by at least 50° C., at least 100° C. or at least 150° C. The second temperature in some embodiments ranges from 100° C. to 800° C. or from 200° C. to 500° C. While not limited to any particular theory, it is believed that the heating facilitates physical desorption of the oligomers from the catalyst 104 along with some cracking of the oligomers.

A carrier gas flowing through the inlet 101 sweeps the oligomers out of the reactor 100 as the product desorbs from the catalyst 104. While the carrier gas may be the same as the feed stream, some embodiments use a different composition for the carrier gas such that the feed stream or the olefins are stopped from passing into contact with the catalyst 104 during the recovery step. For example, steam may function as the carrier gas and also supply the heat needed to reach the second temperature.

In some embodiments, the heating of the catalyst 104 releases the oligomers from the catalyst and into a gas phase. The oligomers released into the gas phase may include C5-C21 hydrocarbons that remained in liquid and solid phases on the catalyst 104 during the passing of the feed stream into contact with the catalyst 104. Effluent from the reactor 100 that exits through the outlet 102 during the recovery step includes the carrier gas along with the oligomers. Separation of the effluent removes the carrier gas and fractionates the products into gasoline, distillate, lube oil and wax ranges for inclusion in respective blend pools.

FIG. 3 illustrates a system schematic depicting operation of the reactor 100 in a regeneration step and the catalyst 104 in a deactivated state as depicted by dot filled circles. For some embodiments, regeneration of the catalyst 104 following the recovery step shown in FIG. 2 occurs in situ within the reactor 100. The regeneration enables further recovering of upgraded products by continuous cycling through each of the steps as described herein.

The regeneration of the catalyst 104 occurs by flowing regeneration gases in sequence through the inlet 101 and into contact with the catalyst 104 inside the reactor 100. The regeneration gases include oxidants (e.g., oxygen or air) introduced to burn coke deposited on the catalyst 104 followed by reducing agents (e.g., carbon monoxide, hydrogen or light hydrocarbons) to reduce active metals of the catalyst 104 to lower oxidation states. In some embodiments, the regeneration of the catalyst 104 includes calcination of the catalyst 104 in air or oxygen atmosphere at 200° C. to 650° C., purging with an inert gas such as steam, nitrogen, argon or carbon dioxide at 200° C. to 500° C. and reduction of the catalyst 104 in carbon monoxide, ethylene, propylene, hydrogen or mixtures thereof at 200° C. to 500° C.

Each following example is provided by way of explanation of the invention, one of many embodiments of the invention, and the following examples should not be read to limit, or define, the scope of the invention.

### Example 1

### Catalyst A

Preparation of Catalyst A was done by dissolving 11.2 grams (g) Cr(NO₃)₃.9H₂O in 43 milliliters (mL) deionized water to form a solution. Then, 40 g of zeolite (SiO₂/Al₂O₃=80) was put into the solution to provide a support for the chromium. After mixing and wetting, an impregnated sample was dried at 110° C. in air for 24 hours and then calcined in air at 550° C. for 5 hours.

### Example 2

### Catalyst B

Preparation of Catalyst B was done by dissolving 3.1 g Ni(NO₃)₂.6H₂O in 42 mL deionized water to form a solution. Then, 40 g of silica beads (surface area=370 meters square per gram (m²/g), pore volume=1.08 mL/g) was put into the solution to provide a support for the nickel. After mixing and wetting, an impregnated sample was dried at 110° C. in air for 24 hours and then calcined in air at 550° C. for 16 hours.

### Example 3

### Catalyst C

Preparation of Catalyst C was done by dissolving 9.5 g Ni(NO₃)₂.6H₂O in 39 mL deionized water to form a solution. Then, 40 g of silica beads (surface area=370 m²/g, pore volume=1.08 mL/g) was put into the solution to provide a support for the nickel. After mixing and wetting, an impregnated sample was dried at 110° C. in air for 24 hours and then calcined in air at 550° C. for 16 hours.

### Example 4

### Oligomerization and Recovery Run with Catalyst A—Rich Olefin Feed

A stainless steel tubular reactor (1.27 centimeter inner diameter) was loaded with 8 g of the Catalyst A. The reactor was heated up to 500° C. and purged with nitrogen for 30 minutes (min). The catalyst was then reduced in carbon monoxide at 350° C. for 60 min. After cooling down to 150° C. in carbon monoxide flow, the reactor was pressurized up to 345 kilopascals for an oligomerization and adsorption operation with 67% ethylene and 33% nitrogen at a flow rate of 300 ml/min for 20 min. Ethylene flow was then stopped. In a subsequent recovery operation, the reactor temperature was increased to 490° C.

An effluent gas condenser collected 7.6 g liquid by completion of the recovery operation. The liquid yield was 35.8%. Simulated distillation indicated that the liquid was composed of 55.5 weight percent (wt. %) C₅-C₁₁, 34.8 wt. % C₁₂-C₂₁ and 8.6 wt. % C₂₂₊ hydrocarbons.

### Example 5

### Oligomerization and Recovery Run with Catalyst A—Lean Olefin Feed

As in Example 4, the reactor was loaded with the Catalyst A, which was then reduced. The reactor at 150° C. was pressurized up to 345 kilopascals for an oligomerization and adsorption operation with 33% ethylene and 67% nitrogen at a flow rate of 300 ml/min for 60 min. Ethylene flow was then stopped. In a subsequent recovery operation, the reactor temperature was increased to 350° C.

The effluent gas condenser collected 2.2 g liquid by completion of the recovery operation. The liquid yield was 27.4%. Simulated distillation indicated that the liquid was composed of 54.3 wt. % C₅-C₁₁, 43.0 wt. % C₁₂-C₂₁ and 2.2 wt. % C₂₂₊ hydrocarbons.

### Example 6

### Oligomerization and Recovery Run with Catalyst B

The Catalyst B was loaded into the reactor and reduced as in Example 4. During an oligomerization and adsorption operation with the reactor at 150° C., 75% ethylene and 25% nitrogen flowed at a rate of 200 ml/min for 70 min. Ethylene flow was then stopped. In a subsequent recovery operation, the reactor temperature was increased to 500° C.

Upon completion of the recovery operation, the effluent gas condenser collected 7.2 g wax. The wax yield was 54.9%. Simulated distillation indicated that the wax was composed of 8.1 wt. % C₅-C₁₁, 23.0 wt. % C₁₂-C₂₁ and 68.9 wt. % C₂₂₊ hydrocarbons.

### Example 7

### Oligomerization and Recovery Run with Catalyst C

The Catalyst C was loaded into the reactor and reduced as in Example 4. During an oligomerization and adsorption operation with the reactor at 150° C., 75% ethylene and 25% nitrogen flowed at a rate of 200 ml/min for 80 min. Ethylene flow was then stopped. In a subsequent recovery operation, the reactor temperature was increased to 500° C.

The effluent gas condenser collected 6.4 g wax by completion of the recovery operation. The wax yield was 42.7%. Simulated distillation indicated that the wax was composed of 16.0 wt. % C₅-C₁₁, 26.9 wt. % C₁₂-C₂₁ and 57.1 wt. % C₂₂₊ hydrocarbons.

In closing, it should be noted that the discussion of any reference is not an admission that it is prior art to the present invention, especially any reference that may have a publication date after the priority date of this application. At the same time, each and every claim below is hereby incorporated into this detailed description or specification as additional embodiments of the present invention.

Although the systems and processes described herein have been described in detail, it should be understood that various changes, substitutions, and alterations can be made without departing from the spirit and scope of the invention as defined by the following claims. Those skilled in the art may be able to study the preferred embodiments and identify other ways to practice the invention that are not exactly as described herein. It is the intent of the inventors that variations and equivalents of the invention are within the scope of the claims whiles the description, abstract and drawings are not to be used to limit the scope of the invention. The invention is specifically intended to be as broad as the claims below and their equivalents.

1. A method, comprising:
Passing a feed stream containing olefins with each between two and five carbons into contact at a first temperature with an oligomerization catalyst to form oligomers of the olefins; and
Recovering the oligomers by heating the catalyst to a second temperature higher than the first temperature such that the oligomers adsorbed on the catalyst when the catalyst and the olefins are contacted at the first temperature are released from the catalyst.

2. The method of claim 1, wherein the passing of the feed stream into contact with the catalyst is stopped during the recovering of the oligomers.

3. The method of claim 1, wherein the recovering of the oligomers includes introducing steam into contact with the catalyst.

4. The method of claim 1, wherein the heating of the catalyst releases the oligomers from the catalyst into a gas phase.

5. The method of claim 1, wherein the heating of the catalyst releases into a gas phase the oligomers including gasoline, distillate, lube oil and wax that remained in liquid and solid phases on the catalyst during the passing of the feed stream into contact with the catalyst.

6. The method of claim 1, wherein the second temperature is at least 50° C. higher than the first temperature.

7. The method of claim 1, wherein the feed stream contains greater than 15% by volume methane and less than 75% by volume of olefins that are at least one of ethylene, propylene and butylenes.

8. The method of claim 1, further comprising separating the oligomers into gasoline and distillate range products after being released from the catalyst during the recovering.

9. The method of claim 1, further comprising regenerating the catalyst in situ after the recovering of the olefins, wherein the regenerating includes oxidation and reduction of the catalyst.

10. The method of claim 1, further comprising contacting the catalyst with oxidants to burn coke deposits remaining after recovering the oligomers and then with reducing agents to lower oxidation states of metals in the catalyst.

11. The method of claim 1, wherein the olefins include ethylene.

12. The method of claim 1, wherein the feed stream is in a gas phase when contacted with the catalyst.

13. The method of claim 1, wherein the feed stream is in a gas phase when contacted with the catalyst and is formed of dilute ethylene.

14. The method of claim 1, wherein the oligomerization catalyst is formed of at least one of chromium, iron, cobalt and nickel on a solid support.

15. The method of claim 1, wherein the passing of the feed stream into contact with the catalyst is at a pressure below 1400 kilopascals and the first temperature is between 20° C. and 300° C.

16. The method of claim 1, wherein the second temperature is between 200° C. and 500° C.

17. The method of claim 1, wherein a porous silica support impregnated with chromium forms the catalyst, the olefins include ethylene, the first temperature is 150° C., the passing of the feed stream into contact with the catalyst is at 345 kilopascals and the second temperature is 500° C.

18. A method, comprising:
contacting an oligomerization catalyst formed of at least one of chromium and nickel on a solid support with a fuel gas containing greater than 15% by volume methane and less than 75% by volume of olefins that are at least one of ethylene, propylene and butylenes, wherein the contacting is at a pressure below 1400 kilopascals and at a first temperature between 20° C. and 300° C. and oligomers of the olefins produced from the contacting are adsorbed on the catalyst; and
heating the catalyst to a second temperature between 200° C. and 500° C. and at least 50° C. higher than the first temperature, wherein the heating desorbs the oligomers that are then separated into gasoline and distillate range products.

19. The method of claim 18, further comprising regenerating the catalyst in situ after heating the catalyst to the second temperature, wherein the regenerating includes oxidation and reduction of the catalyst.

20. A system, comprising:
a reactor containing an oligomerization catalyst and coupled to a feed of olefins selected from at least one of ethylene, propylene and butylenes, wherein the reactor is configured to cycle between an oligomerization and adsorption operation in which the olefins and the catalyst are in fluid communication for contact at a first temperature and a recovery operation in which heating to a second temperature higher than the first temperature desorbs from the catalyst oligomers of the olefins.
